(19) United States
(12) Patent Application Publication
(10) Pub. No.: US 2024/0197632 A1
(43) Pub. Date: Jun. 20, 2024

(54) WATER SOLUBLE COMPLEX COMPOSITIONS OF IBUPROFEN

(71) Applicant: VETERINARY PHARMACY CORPORATION, Saint Peter, MN (US)

(72) Inventor: Patrick John Smith, Saint Peter, MN (US)

(21) Appl. No.: 18/425,507

(22) Filed: Jan. 29, 2024

Related U.S. Application Data
(60) Provisional application No. 63/385,872, filed on Dec. 2, 2022.

(51) Int. Cl. A61K 9/10; A61K 31/192; A61K 47/02; A61K 47/12; A61K 47/18; A61K 47/22

(52) U.S. Cl. CPC A61K 9/10; A61K 31/192; A61K 47/02; A61K 47/12; A61K 47/183; A61K 47/22

(57) ABSTRACT

The disclosure herein describes solubilizing an ibuprofen in water forming a complex with a hydrotrope and controlling the pH of the water or water-based solution. The methods for solubilizing ibuprofen in water are described. These methods can include forming a colloidal suspension of the ibuprofen, and then forming an aqueous colloidal suspension from the colloidal suspension.

### FIELD

The present disclosure relates generally to formulating a water insoluble compound to be in a soluble form for treatment of animals.

### BACKGROUND

Ibuprofen (2-(4-Isobutylphenyl)propanoic acid) is a commonly used nonsteroidal antiinflammatory (NSAID) drug which is considered to be among the safest NSAIDs and is generally well tolerated. It is generally known that ibuprofen is insoluble in water, having a general solubility property of 21 mg/L (0.0021% wt/vol). Ibuprofen has the molecular formula of C₁₃H₁₈O₂ and the following structure:

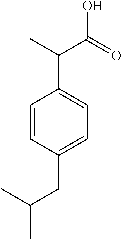

### SUMMARY

Any or all portion(s) of any of the embodiments disclosed herein may be combined with any other portion(s) of any embodiment.

Some of the embodiments disclosed herein relate to methods for solubilizing a water insoluble compound in water or water-based colloidal suspension.

Some of the embodiments disclosed herein relate to methods for solubilizing ibuprofen in water or water-based colloidal suspension.

Some embodiments of a method for solubilizing ibuprofen in water comprises obtaining the ibuprofen; obtaining the water; obtaining a hydrotrope; and forming a colloidal suspension of ibuprofen, wherein the forming of the colloidal suspension of ibuprofen comprises mixing the ibuprofen and the hydrotrope in the water; and forming an aqueous colloidal suspension of ibuprofen from the colloidal suspension of ibuprofen, wherein the forming of the aqueous colloidal suspension of ibuprofen comprises obtaining a base; and adding an effective amount of the base to the colloidal suspension of ibuprofen to change the pH of the colloidal suspension of ibuprofen to be greater than 7.

In some embodiments, the solubilization of ibuprofen includes at least one of niacinamide, glycine, sodium salicylate, proline, arginine, or any combination thereof.

In some embodiments of the method, the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

In some embodiments of the method, the hydrotrope comprises an alkali metal salt. In some embodiments of the method, the alkali metal salt comprises a lithium salt. In some embodiments of the method, the alkali metal salt comprises a sodium salt. In some embodiments of the method, the alkali metal salt comprises a potassium salt. In some embodiments of the method, the alkali metal salt comprises a calcium salt.

In some embodiments of the method, the base comprises a non-conventional Lewis base.

In some embodiments of the method, the base comprises a non-conventional hydroxide Lewis base.

In some embodiments of the method, the base comprises at least one of sodium hydroxide, potassium hydroxide, strontium hydroxide, barium hydroxide, rubidium hydroxide, calcium hydroxide, ammonium hydroxide, magnesium hydroxide, lithium hydroxide, cesium hydroxide, DMSO, DMA, Trimethylphosphine, EtOAC, arginine, ammonia, trimethyl ammonia, pyridine, methylamine, alanine, or a combination thereof.

In some embodiments of the method, the forming of the aqueous colloidal suspension of ibuprofen further comprises obtaining a pH buffer; and adding the pH buffer to the colloidal suspension of ibuprofen.

In some embodiments of the method, the aqueous colloidal suspension of ibuprofen includes: the pH buffer present in an amount of 0.001 g/ml to 0.31 g/ml of the aqueous colloidal suspension of ibuprofen.

In some embodiments of the method, the aqueous colloidal suspension of ibuprofen includes the ibuprofen present at least in an amount of 0.000021 g/ml of the aqueous colloidal suspension of ibuprofen, the hydrotrope being present in an amount of 0.01 g/ml to 0.6 g/ml of the aqueous colloidal suspension of ibuprofen; and the pH buffer present in an amount of 0.001 g/ml to 0.31 g/ml of the aqueous colloidal suspension of ibuprofen.

In some embodiments of the method, the pH buffer comprises at least one of glycine, arginine, or a combination thereof.

In some embodiments of the method, the pH buffer comprises at least one of ACES, ADA, ammonium hydroxide, 2-amino-2-methyl-1-propanol, AMPD (2 amino-2-methyl-1,3-propanediol), AMPSO, BES, BICINE, bis-tris, bis-tris propane, borate, boric acid, CABs, Cacodylate, CAPS, CAPSO, Carbonate (Salts included), CHES, Citrate (Salts included), DIPS, EPPS, HEPPS, Ethanolamine, Glycine, glycylglycine, HEPBS, HEPES, HEPPSO, histidine, hydrazine, imidazole, maleate, MES, methylamine, MOBS, MOPS, MOPSO, phosphate (Salts included), piperazine, piperidine, PIPES, POPSO, pyrophosphate, TABS, TAPS, TAPSO, taurine, TES, tricine, triethanolamine, trizma, or a combination thereof.

In some embodiments of the method, the hydrotrope is sodium salicylate in an amount of 0.01 g/ml to 0.6 g/ml of the aqueous colloidal suspension of ibuprofen; and the pH buffer is in an amount of at least 0.001 g/ml to 0.31 g/ml of the aqueous colloidal suspension of ibuprofen.

In some embodiments of the method, the hydrotrope is sodium salicylate in an amount of at least 0.08 g/ml of the aqueous colloidal suspension of ibuprofen; and the pH buffer is in an amount of at least 0.02 g/ml of the aqueous colloidal suspension of ibuprofen.

In some embodiments of the method, the hydrotrope is niacinamide in an amount of 0.001 g/ml to 0.60 g/ml of the aqueous colloidal suspension of ibuprofen; and the pH buffer is in an amount of at least 0.001 g/ml to 0.04 g/ml of the aqueous colloidal suspension of ibuprofen.

In some embodiments, the pH buffer includes glycine. In some embodiments, the pH buffer is glycine. In some embodiments, the pH buffer does not include glycine. In some embodiments, the pH buffer is not glycine.

In some embodiments, the pH buffer includes arginine. In some embodiments, the pH buffer is arginine. In some embodiments, the pH buffer does not include arginine. In some embodiments, the pH buffer is not arginine.

In some embodiments of the method, the obtaining the ibuprofen comprises obtaining a tablet comprising the ibuprofen as an active pharmaceutical ingredient (API).

In some embodiments, a method for treating illness in an animal comprises forming a medicated water-based colloidal suspension, wherein the forming the medicated water-based colloidal suspension comprises the method of solubilizing ibuprofen in water; and providing the medicated water-based colloidal suspension to the animal.

In some embodiments of the method, the animal is at least one of a pig, poultry, a cattle, a sheep, a horse, a dog, or a cat.

In some embodiments of the method, the animal is a farm animal.

In some embodiments, an aqueous colloidal suspension of ibuprofen comprises water; ibuprofen, wherein the ibuprofen is present in an amount of 0.000021 to 0.031 g/ml of the water; a hydrotrope, wherein the hydrotrope is present in an amount of 0.001 to 0.6 g/ml of the water; and a base, wherein the aqueous colloidal suspension of ibuprofen has a pH of greater than 7.

In some embodiments, the aqueous colloidal suspension of ibuprofen has a ibuprofen concentration of 0.0021%-31.5%. In some embodiments, the aqueous colloidal suspension of ibuprofen has a ibuprofen concentration of 2.0%-12%. In some embodiments, the aqueous colloidal suspension of ibuprofen has a ibuprofen concentration of 3.0%-12%. In some embodiments, the aqueous colloidal suspension of ibuprofen has a ibuprofen concentration of 4.0%-12%. In some embodiments, the aqueous colloidal suspension of ibuprofen has a ibuprofen concentration of 5.0%-12%. In some embodiments, the aqueous colloidal suspension of ibuprofen has a ibuprofen concentration of 6.0%-12%. In some embodiments, the aqueous colloidal suspension of ibuprofen has a ibuprofen concentration of 7.0%-12%. In some embodiments, the aqueous colloidal suspension of ibuprofen has a ibuprofen concentration of 8.0%-12%. In some embodiments, the aqueous colloidal suspension of ibuprofen has a ibuprofen concentration of 9.0%-12%. In some embodiments, the aqueous colloidal suspension of ibuprofen has a ibuprofen concentration of 10.0%-12%. In some embodiments, the aqueous colloidal suspension of ibuprofen has a ibuprofen concentration of 11.0%-12%. In some embodiments, the aqueous colloidal suspension of ibuprofen has a ibuprofen concentration of 1.0%-2.0%. In some embodiments, the aqueous colloidal suspension of ibuprofen has a ibuprofen concentration of 1.0%-3.0%. In some embodiments, the aqueous colloidal suspension of ibuprofen has a ibuprofen concentration of 1.0%-4.0%. In some embodiments, the aqueous colloidal suspension of ibuprofen has a ibuprofen concentration of 1.0%-5.0%. In some embodiments, the aqueous colloidal suspension of ibuprofen has a ibuprofen concentration of 1.0%-6.0%. In some embodiments, the aqueous colloidal suspension of ibuprofen has a ibuprofen concentration of 1.0%-7.0%. In some embodiments, the aqueous colloidal suspension of ibuprofen has a ibuprofen concentration of 1.0%-8.0%. In some embodiments, the aqueous colloidal suspension of ibuprofen has a ibuprofen concentration of 1.0%-9.0%. In some embodiments, the aqueous colloidal suspension of ibuprofen has a ibuprofen concentration of 1.0%-10%. In some embodiments, the aqueous colloidal suspension of ibuprofen has a ibuprofen concentration of 1.0%-11%. In some embodiments, the aqueous colloidal suspension of ibuprofen has a ibuprofen concentration of 1.0%-12%. In some embodiments, the aqueous colloidal suspension of ibuprofen has a ibuprofen concentration of 2.0%-3.0%. In some embodiments, the aqueous colloidal suspension of ibuprofen has a ibuprofen concentration of 2.0%-4.0%. In some embodiments, the aqueous colloidal suspension of ibuprofen has a ibuprofen concentration of 2.0%-5.0%. In some embodiments, the aqueous colloidal suspension of ibuprofen has a ibuprofen concentration of 2.0%-6.0%. In some embodiments, the aqueous colloidal suspension of ibuprofen has a ibuprofen concentration of 2.0%-7.0%. In some embodiments, the aqueous colloidal suspension of ibuprofen has a ibuprofen concentration of 2.0%-8.0%. In some embodiments, the aqueous colloidal suspension of ibuprofen has a ibuprofen concentration of 2.0%-9.0%. In some embodiments, the aqueous colloidal suspension of ibuprofen has a ibuprofen concentration of 2.0%-10%. In some embodiments, the aqueous colloidal suspension of ibuprofen has a ibuprofen concentration of 2.0%-11%. In some embodiments, the aqueous colloidal suspension of ibuprofen has a ibuprofen concentration of 2.0%-12%. In some embodiments, the aqueous colloidal suspension of ibuprofen has a ibuprofen concentration of 3.0%-4.0%. In some embodiments, the aqueous colloidal suspension of ibuprofen has a ibuprofen concentration of 3.0%-5.0%. In some embodiments, the aqueous colloidal suspension of ibuprofen has a ibuprofen concentration of 3.0%-6.0%. In some embodiments, the aqueous colloidal suspension of ibuprofen has a ibuprofen concentration of 3.0%-7.0%. In some embodiments, the aqueous colloidal suspension of ibuprofen has a ibuprofen concentration of 3.0%-8.0%. In some embodiments, the aqueous colloidal suspension of ibuprofen has a ibuprofen concentration of 3.0%-9.0%. In some embodiments, the aqueous colloidal suspension of ibuprofen has a ibuprofen concentration of 3.0%-10%. In some embodiments, the aqueous colloidal suspension of ibuprofen has a ibuprofen concentration of 3.0%-11%. In some embodiments, the aqueous colloidal suspension of ibuprofen has a ibuprofen concentration of 3.0%-12%. In some embodiments, the aqueous colloidal suspension of ibuprofen has a ibuprofen concentration of 4.0%-5.0%. In some embodiments, the aqueous colloidal suspension of ibuprofen has a ibuprofen concentration of 4.0%-6.0%. In some embodiments, the aqueous colloidal suspension of ibuprofen has a ibuprofen concentration of 4.0%-7.0%. In some embodiments, the aqueous colloidal suspension of ibuprofen has a ibuprofen concentration of 4.0%-8.0%. In some embodiments, the aqueous colloidal suspension of ibuprofen has a ibuprofen concentration of 4.0%-9.0%. In some embodiments, the aqueous colloidal suspension of ibuprofen has a ibuprofen concentration of 4.0%-10%. In some embodiments, the aqueous colloidal suspension of ibuprofen has a ibuprofen concentration of 4.0%-11%. In some embodiments, the aqueous colloidal suspension of ibuprofen has a ibuprofen concentration of 4.0%-12%. In some embodiments, the aqueous colloidal suspension of ibuprofen has a ibuprofen concentration of 5.0%-6.0%. In some embodiments, the aqueous colloidal suspension of ibuprofen has a ibuprofen concentration of 5.0%-7.0%. In some embodiments, the aqueous colloidal suspension of ibuprofen has a ibuprofen concentration of 5.0%-8.0%. In some embodiments, the aqueous colloidal suspension of ibuprofen has a ibuprofen concentration of 5.0%-9.0%. In some embodiments, the aqueous colloidal suspension of ibuprofen has a ibuprofen concentration of 5.0%-10%. In some embodiments, the aqueous colloidal suspension of ibuprofen has a ibuprofen concentration of 5.0%-11%. In some embodiments, the aqueous colloidal suspension of ibuprofen has a ibuprofen concentration of 5.0%-12%. In some embodiments, the aqueous colloidal suspension of ibuprofen has a ibuprofen concentration of 6.0%-7.0%. In some embodiments, the aqueous colloidal suspension of ibuprofen has a ibuprofen concentration of 6.0%-8.0%. In some embodiments, the aqueous colloidal suspension of ibuprofen has a ibuprofen concentration of 6.0%-9.0%. In some embodiments, the aqueous colloidal suspension of ibuprofen has a ibuprofen concentration of 6.0%-10%. In some embodiments, the aqueous colloidal suspension of ibuprofen has a ibuprofen concentration of 6.0%-11%. In some embodiments, the aqueous colloidal suspension of ibuprofen has a ibuprofen concentration of 6.0%-12%. In some embodiments, the aqueous colloidal suspension of ibuprofen has a ibuprofen concentration of 7.0%-8.0%. In some embodiments, the aqueous colloidal suspension of ibuprofen has a ibuprofen concentration of 7.0%-9.0%. In some embodiments, the aqueous colloidal suspension of ibuprofen has a ibuprofen concentration of 7.0%-10%. In some embodiments, the aqueous colloidal suspension of ibuprofen has a ibuprofen concentration of 7.0%-11%. In some embodiments, the aqueous colloidal suspension of ibuprofen has a ibuprofen concentration of 7.0%-12%. In some embodiments, the aqueous colloidal suspension of ibuprofen has a ibuprofen concentration of 8.0%-9.0%. In some embodiments, the aqueous colloidal suspension of ibuprofen has a ibuprofen concentration of 8.0%-10%. In some embodiments, the aqueous colloidal suspension of ibuprofen has a ibuprofen concentration of 8.0%-11%. In some embodiments, the aqueous colloidal suspension of ibuprofen has a ibuprofen concentration of 8.0%-12%. In some embodiments, the aqueous colloidal suspension of ibuprofen has a ibuprofen concentration of 9.0%-10%. In some embodiments, the aqueous colloidal suspension of ibuprofen has a ibuprofen concentration of 9.0%-11%. In some embodiments, the aqueous colloidal suspension of ibuprofen has a ibuprofen concentration of 9.0%-12%. In some embodiments, the aqueous colloidal suspension of ibuprofen has a ibuprofen concentration of 10%-11%. In some embodiments, the aqueous colloidal suspension of ibuprofen has a ibuprofen concentration of 10%-12%. In some embodiments, the aqueous colloidal suspension of ibuprofen has a ibuprofen concentration of 11%-12%.

In some embodiments of the aqueous colloidal suspension of ibuprofen, the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

In some embodiments of the aqueous colloidal suspension of ibuprofen, the base comprises sodium hydroxide.

In some embodiments, a method for solubilizing ibuprofen in a stock colloidal suspension comprises obtaining the ibuprofen; obtaining the stock colloidal suspension; obtaining a hydrotrope; forming a colloidal suspension of ibuprofen, wherein the forming of the colloidal suspension of ibuprofen comprises mixing the ibuprofen and the hydrotrope in the stock colloidal suspension; and forming a medicated stock colloidal suspension from the colloidal suspension of ibuprofen, wherein the forming of the medicated stock colloidal suspension comprises obtaining a base; and adding an effective amount of the base to the colloidal suspension of ibuprofen to change the pH of the colloidal suspension of ibuprofen to prevent precipitation during the compounding process, and for solubilization to occur.

In some embodiments, the method for solubilizing ibuprofen in a stock colloidal suspension comprises adding an effective amount of the base to the colloidal suspension of ibuprofen to change the pH of the colloidal suspension of ibuprofen to at least 6.0 to prevent precipitation during the compounding process.

In some embodiments, the pH of the colloidal suspension of ibuprofen must be above 7.0 for solubilization to occur.

In some embodiments, the method further comprising mixing the medicated stock colloidal suspension with water.

In some embodiments, a method for treating illness in an animal comprises forming a medicated water-based colloidal suspension, wherein the forming the medicated water-based colloidal suspension comprises the method of solubilizing ibuprofen in a stock colloidal suspension; and providing the medicated water-based colloidal suspension to the animal.

In some embodiments of the method, the animal is at least one of a pig, poultry, a cattle, a sheep, a horse, a dog, or a cat.

In some embodiments, a method for solubilizing ibuprofen in water comprises obtaining the ibuprofen; obtaining the water; obtaining a hydrotrope; and forming a colloidal suspension of ibuprofen, wherein the forming of the colloidal suspension of ibuprofen comprises mixing the ibuprofen and the hydrotrope in the water; and forming an aqueous colloidal suspension of ibuprofen from the colloidal suspension of ibuprofen, wherein the forming of the aqueous colloidal suspension of ibuprofen comprises obtaining a base; and adding an effective amount of the base to the colloidal suspension of ibuprofen to change the pH of the colloidal suspension of ibuprofen to be greater than 7.

In some embodiments, the medicated stock colloidal suspension is mixed with water at a mixing ratio of 1:50 (the medicated stock colloidal suspension:water). In some embodiments, the medicated stock colloidal suspension is mixed with water at a mixing ratio of 1:64 (the medicated stock colloidal suspension:water). In some embodiments, the medicated stock colloidal suspension is mixed with water at a mixing ratio of 1:100 (the medicated stock colloidal suspension:water). In some embodiments, the medicated stock colloidal suspension is mixed with water at a mixing ratio of 1:128 (the medicated stock colloidal suspension:water). In some embodiments, the medicated stock colloidal suspension is mixed with water at a mixing ratio of 1:200 (the medicated stock colloidal suspension:water). In some embodiments, the medicated stock colloidal suspension is mixed with water at a mixing ratio of 1:500 (the medicated stock colloidal suspension:water).

### BRIEF DESCRIPTION OF THE DRAWINGS

References are made to the accompanying drawings that form a part of this disclosure and that illustrate embodiments in which the systems and methods described in this Specification can be practiced.

FIG. 1 is a schematic diagram and flowchart showing an embodiment for providing a medicated water solution.

### DETAILED DESCRIPTION

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

The embodiments disclosed herein are directed towards formulating a water insoluble compound (e.g., ibuprofen) to be in a soluble form, via forming a complex composition with a hydrotrope and affecting the pH of the mixture to form a colloidal suspension. The resultant colloidal suspension can be further mixed with water or water-based solution (e.g., stock) for providing the resultant medicated water or medicated water-based solution to treat animals.

Colloidal suspension, as used herein, means a mixture or a complex composition in which a first substance is divided into minute particles (called colloidal particles) and dispersed throughout a second substance. The first and second substances are present as larger particles than those found in a traditional solution, but these larger particles are too small to be seen with a microscope. When the colloidal suspension is in water, it is called an aqueous colloidal suspension. There are generally no strict boundaries on the size of colloidal particles, but they can be from 10-9 m to 10-6 m in size. A colloidal suspension is not a true solution but it is also not a “true suspension” either because the colloidal particles do not settle out like a true suspension will over time. The colloidal suspension will also display a Tyndal effect phenomenon and Brownian motion. As used herein, the term “solution” is used to describe a colloidal suspension (and for clarity, the term “true solution” is used for a type of mixture which does not include a colloidal suspension). The colloidal suspension can be formed via use of hydrotropes.

Hydrotropes (also called “hydrotropic agent,” e.g., niacinamide) have a property to increase the solubility of insoluble molecules. A common feature of many hydrotropes is a small aromatic ring that allows it to have this effect. The interaction may not be entirely colloidal. Instead, the hydrotropic agent has a weak interaction with a solute molecule (which may be generally insoluble) that allow a complex to form. This complex is what becomes soluble. The efficiency of the hydrotropic agent depends on a balance between its hydrophilic and hydrophobic pieces. The larger the hydrophobic part of the agent the better its efficiency. Another common feature of hydrotropic agents is their own high solubility (e.g., in water). Other mechanisms that can be important to better efficiency is self-aggregation potential, structure breaker, and structure maker, and ability to form a micelle like structure. There can also be an importance of a minimum hydrotropic agent concentration needed to achieve solubility of a solute. This minimum concentration is a concentration at which the hydrotrope aggregates. At higher concentrations, the hydrotropes begin to decrease the cloud point of the solution.

In an example, 24.2 g of ibuprofen was obtained and mixed with 100 ml of water, with an addition of 28.1 g of niacinamide, with an addition of 2 g of glycine buffer. The mixture was agitated, forming a suspension. The initial pH of the mixture was noted to be at ˜6.9-7.0. Upon adding 5.08 g sodium hydroxide to raise the pH to 11.0. The stability of this clear solution was tested (to see whether it would be brought out of solution) by adding water from various sources (e.g., tap water, distilled water, etc.). Surprisingly, the addition or disturbance using more water did not cause any precipitation of the product.

The following are some example formulas for the ibuprofen complex water-soluble products. Target pH for all of the Examples was greater than 7.0:

### 1) EXAMPLE 1

### Ibuprofen Solution Example 1

a. ibuprofen 24.2 g
b. niacinamide 28.1 g
c. glycine buffer 2 gl
d. NaOH 5.08 g
e. H₂O q.s. 100 ml

### 2) EXAMPLE 2

### Ibuprofen Solution

a. ibuprofen 21.4 g
b. niacinamide 11.5 gl
c. sodium salicylate 13.2 g
d. NaOH 4.5 g
e. glycine buffer 2 g/100 ml
f. H₂O q.s. 150 ml

### 3) EXAMPLE 3

### Ibuprofen Solution

a. ibuprofen 21.4 g
b. niacinamide 25.9 g
c. glycine buffer 3 g
d. NaOH 4.6 g
e. H₂O q.s. 100 ml

NOTE: This Example showed extreme stability of 21.4% ibuprofen solution at a pH of 9.5. The hydrotrope ratio was also confirmed. These results have been verified and has been confirmed for repeatability. Surprisingly, after 508 days, this Example showed that ibuprofen remained in solution and within potency specification.

### 4) EXAMPLE 4

### Ibuprofen Solution

a. ibuprofen 25.7 g
b. proline 25.2 g
c. niacinamide 5.4 g
d. glycine buffer 3 g
e NaOH 5.7 g
f. H₂O q.s. 100 ml

### 5) EXAMPLE 5

### Ibuprofen Solution

a. Ibuprofen 26.4 g
b. arginine 26.3 g
c. H₂O q.s. 100 ml

### 6) EXAMPLE 6

### Ibuprofen Solution

a. Ibuprofen 31.5 g
b. arginine 31.5 g
c. H₂O q.s. 100 ml

NOTE: Example 5 and Example 6 surprisingly showed ibuprofen solution formation. Surprisingly, after 512 days, Example 5 showed that ibuprofen remained in solution and within potency specification.

Target Market Example: Livestock/animal(s) (e.g., pig, poultry, cattle, sheep, horse, dog, cat, etc.).

There are estimated to be about 6.41 million sows in the U.S. Market. Cross-sectional studies show that the within-herd prevalence of sow lameness is quite high and may range from 8.8% to 16.9%. This equates to a potential market of 564,000 to 1,083,000 animals requiring treatment at some point.

There are roughly 75 million hogs in the United States. Studies have shown anywhere from the percentage seen in sows to up to 40% or more of growing herd can experience lameness. This would equate to up to 30 million hogs potentially experiencing this debilitating disease at some point and requiring treatment.

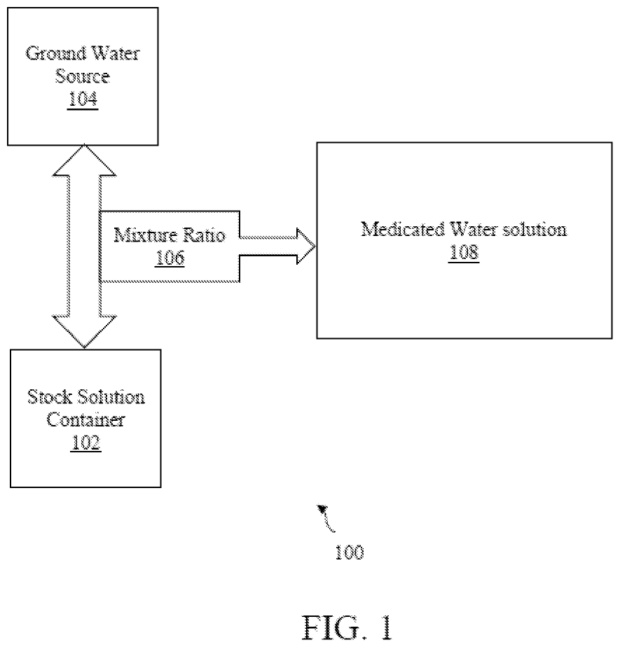

FIG. 1 shows a schematic diagram showing an embodiment of a treatment administration system and process. According to this system and process, ibuprofen is provided for animal(s) in a diluted form, delivered via a water medication system 100. A container with concentrated ibuprofen solution or dry powder packet blend is added to stock solution container 102. Medication is diluted in X gallons of Stock Solution. This product is mixed with a ground water source 104 at a dilution rate or ratio 106 (e.g., 1 gallon Stock Solution:128 gallons Water). The resulting Medicated Water solution 108 is provided to the animal(s). In some embodiments, the Stock Solution to Water mixing ratio is 1:50, 1:64, 1:100, 1:128, 1:200, or 1:500.

### ASPECTS

Any of the Aspects (or any portions of the Aspects) provided below can be combined with any one or more of the other Aspects.

Aspect 1. In some aspects, the techniques described herein relate to a method for solubilizing ibuprofen in water, including: obtaining the ibuprofen; obtaining the water; obtaining a hydrotrope; and forming a colloidal suspension of ibuprofen, wherein the forming of the colloidal suspension of ibuprofen includes: mixing the ibuprofen and the hydrotrope in the water; and forming an aqueous colloidal suspension of ibuprofen from the colloidal suspension of ibuprofen, wherein the forming of the aqueous colloidal suspension of ibuprofen includes: obtaining a base; and adding an effective amount of the base to the colloidal suspension of ibuprofen to change a pH of the colloidal suspension of ibuprofen to be greater than 7.

Aspect 2. In some aspects, the techniques described herein relate to a method, wherein the hydrotrope includes at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

Aspect 3. In some aspects, the techniques described herein relate to a method, wherein the hydrotrope includes an alkali metal salt.

Aspect 4. In some aspects, the techniques described herein relate to a method, wherein the hydrotrope includes at least one of sodium benzene sulfonate, sodium benzene di-sulfonate, sodium cinnamate, sodium 3-hydroxy-2-naphthoate, sodium para toluene sulfonate, sodium cumene sulfonate, N,N-diethylnicotinamide, N,N-dimethyl benzamide, paraaminobenzoic acid hydrochloride, procaine hydrochloride, sodium alkanoate, urea, N,N-dimethyl urea, resorcinol, pyrogallol, catechol, a,b-napthols, N-diethylnicotinamide (DENA), N,N-dimethylbenzamide (DMBA), sodium xylene sulfonate, ammonium xylene sulfonate, sodium cumene sulfonate, sodium toluene sulfonate, potassium toluene sulfonate, sodium alginate, ibuprofen, arginine, tyrosine, sodium acetate, sodium ascorbate, leucine, valine, glutamine, histidine, asparagine, or a combination thereof.

Aspect 5. In some aspects, the techniques described herein relate to a method, wherein the base includes at least one of sodium hydroxide, potassium hydroxide, strontium hydroxide, barium hydroxide, rubidium hydroxide, calcium hydroxide, ammonium hydroxide, magnesium hydroxide, lithium hydroxide, cesium hydroxide, DMSO, DMA, Trimethylphosphine, EtOAC, arginine, ammonia, trimethyl ammonia, pyridine, methylamine, alanine, or a combination thereof.

Aspect 6. In some aspects, the techniques described herein relate to a Aspect 6. method, wherein the forming of the aqueous colloidal suspension of ibuprofen further includes: obtaining a pH buffer; and adding the pH buffer to the colloidal suspension of ibuprofen.

Aspect 7. In some aspects, the techniques described herein relate to a method, wherein the pH buffer includes at least one of glycine, arginine, or a combination thereof.

Aspect 8. In some aspects, the techniques described herein relate to a method, wherein the pH buffer includes at least one of ACES, ADA, ammonium hydroxide, 2-amino-2-methyl-1-propanol, AMPD (2 amino-2-methyl-1,3-propanediol), AMPSO, BES, BICINE, bis-tris, bis-tris propane, borate, boric acid, CABs, Cacodylate, CAPS, CAPSO, Carbonate (Salts included), CHES, Citrate (Salts included), DIPS, EPPS, HEPPS, Ethanolamine, Glycine, glycylglycine, HEPBS, HEPES, HEPPSO, histidine, hydrazine, imidazole, maleate, MES, methylamine, MOBS, MOPS, MOPSO, phosphate (Salts included), piperazine, piperidine, PIPES, POPSO, pyrophosphate, TABS, TAPS, TAPSO, taurine, TES, tricine, triethanolamine, trizma, or a combination thereof.

Aspect 9. In some aspects, the techniques described herein relate to a method, wherein the obtaining the ibuprofen includes obtaining a tablet including the ibuprofen as an active pharmaceutical ingredient (API).

Aspect 10. In some aspects, the techniques described herein relate to a method for treating illness in an animal, including: forming a medicated water-based colloidal suspension, wherein the forming the medicated water-based colloidal suspension includes: the method 1; and providing the medicated water-based colloidal suspension to the animal.

Aspect 11. In some aspects, the techniques described herein relate to an aqueous colloidal suspension of ibuprofen, including: water; ibuprofen; a hydrotrope; and a base, wherein the aqueous colloidal suspension of ibuprofen has a pH of greater than 7.

Aspect 12. In some aspects, the techniques described herein relate to a method for solubilizing ibuprofen in a stock colloidal suspension, including: obtaining the ibuprofen; obtaining the stock colloidal suspension; obtaining a hydrotrope; forming a colloidal suspension of ibuprofen, wherein the forming of the colloidal suspension of ibuprofen includes: mixing the ibuprofen and the hydrotrope in the stock colloidal suspension; and forming a medicated stock colloidal suspension from the colloidal suspension of ibuprofen, wherein the forming of the medicated stock colloidal suspension includes: obtaining a base; and adding an effective amount of the base to the colloidal suspension of ibuprofen to change a pH of the colloidal suspension of ibuprofen to at least 8.

Aspect 13. In some aspects, the techniques described herein relate to a method for solubilizing ibuprofen in water, including: obtaining the ibuprofen; obtaining the water; obtaining a hydrotrope; and forming a colloidal suspension of ibuprofen, wherein the forming of the colloidal suspension of ibuprofen includes: mixing the ibuprofen and the hydrotrope in the water; and forming an aqueous colloidal suspension of ibuprofen from the colloidal suspension of ibuprofen, wherein the forming of the aqueous colloidal suspension of ibuprofen includes: obtaining a base; and adding an effective amount of the base to the colloidal suspension of ibuprofen to change a pH of the colloidal suspension of ibuprofen to be greater than 7.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

What is claimed is:

1. A method for solubilizing ibuprofen in water, comprising:
obtaining the ibuprofen;
obtaining the water;
obtaining a hydrotrope; and
forming a colloidal suspension of ibuprofen,
wherein the forming of the colloidal suspension of ibuprofen comprises:
mixing the ibuprofen and the hydrotrope in the water; and
forming an aqueous colloidal suspension of ibuprofen from the colloidal suspension of ibuprofen,
wherein the forming of the aqueous colloidal suspension of ibuprofen comprises:
obtaining a base; and
adding an effective amount of the base to the colloidal suspension of ibuprofen to change a pH of the colloidal suspension of ibuprofen to be greater than 7.

2. The method of claim 1, wherein the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

3. The method of claim 1, wherein the hydrotrope comprises an alkali metal salt.

4. The method of claim 1, wherein the hydrotrope comprises at least one of sodium benzene sulfonate, sodium benzene di-sulfonate, sodium cinnamate, sodium 3-hydroxy-2-naphthoate, sodium para toluene sulfonate, sodium cumene sulfonate, N,N-diethylnicotinamide, N,N-dimethyl benzamide, paraaminobenzoic acid hydrochloride, procaine hydrochloride, sodium alkanoate, urea, N,N-dimethyl urea, resorcinol, pyrogallol, catechol, a,b-napthols,N-diethylnicotinamide (DENA), N,N-dimethylbenzamide (DMBA), sodium xylene sulfonate, ammonium xylene sulfonate, sodium cumene sulfonate, sodium toluene sulfonate, potassium toluene sulfonate, sodium alginate, ibuprofen, arginine, tyrosine, sodium acetate, sodium ascorbate, leucine, valine, glutamine, histidine, asparagine, or a combination thereof.

5. The method of claim 1, wherein the base comprises at least one of sodium hydroxide, potassium hydroxide, strontium hydroxide, barium hydroxide, rubidium hydroxide, calcium hydroxide, ammonium hydroxide, magnesium hydroxide, lithium hydroxide, cesium hydroxide, DMSO, DMA, Trimethylphosphine, EtOAC, arginine, ammonia, trimethyl ammonia, pyridine, methylamine, alanine, or a combination thereof.

6. The method of claim 1, wherein the forming of the aqueous colloidal suspension of ibuprofen further comprises:
obtaining a pH buffer; and
adding the pH buffer to the colloidal suspension of ibuprofen.

7. The method of claim 6, wherein the pH buffer comprises at least one of glycine, arginine, or a combination thereof.

8. The method of claim 6, wherein the pH buffer comprises at least one of ACES, ADA, ammonium hydroxide, 2-amino-2-methyl-1-propanol, AMPD (2 amino-2-methyl-1,3-propanediol), AMPSO, BES, BICINE, bis-tris, bis-tris propane, borate, boric acid, CABs, Cacodylate, CAPS, CAPSO, Carbonate (Salts included), CHES, Citrate (Salts included), DIPS, EPPS, HEPPS, Ethanolamine, Glycine, glycylglycine, HEPBS, HEPES, HEPPSO, histidine, hydrazine, imidazole, maleate, MES, methylamine, MOBS, MOPS, MOPSO, phosphate (Salts included), piperazine, piperidine, PIPES, POPSO, pyrophosphate, TABS, TAPS, TAPSO, taurine, TES, tricine, triethanolamine, trizma, or a combination thereof.

9. The method of claim 1, wherein the obtaining the ibuprofen comprises obtaining a tablet comprising the ibuprofen as an active pharmaceutical ingredient (API).

10. A method for treating illness in an animal, comprising:
forming a medicated water-based colloidal suspension,
wherein the forming the medicated water-based colloidal suspension comprises:
the method of solubilizing ibuprofen in water according to claim 1; and
providing the medicated water-based colloidal suspension to the animal.

11. An aqueous colloidal suspension of ibuprofen, comprising:
water;
ibuprofen;
a hydrotrope; and
a base,
wherein the aqueous colloidal suspension of ibuprofen has a pH of greater than 7.

12. The aqueous colloidal suspension of claim 11, wherein the hydrotrope comprises at least one of nicotinamide, niacinamide, caffeine, urea, para amino benzoic acid, tryptophan, proline, phenylalanine, niacin, acetylsalicylic acid, sodium citrate, sodium salicylate, sodium benzoate, or a combination thereof.

13. The aqueous colloidal suspension of claim 11, wherein the hydrotrope comprises at least one of sodium benzene sulfonate, sodium benzene di-sulfonate, sodium cinnamate, sodium 3-hydroxy-2-naphthoate, sodium para toluene sulfonate, sodium cumene sulfonate, N,N-diethylnicotinamide, N,N-dimethyl benzamide, paraaminobenzoic acid hydrochloride, procaine hydrochloride, sodium alkanoate, urea, N,N-dimethyl urea, resorcinol, pyrogallol, catechol, a,b-napthols, N-diethylnicotinamide (DENA), N,N-dimethylbenzamide (DMBA), sodium xylene sulfonate, ammonium xylene sulfonate, sodium cumene sulfonate, sodium toluene sulfonate, potassium toluene sulfonate, sodium alginate, ibuprofen, arginine, tyrosine, sodium acetate, sodium ascorbate, leucine, valine, glutamine, histidine, asparagine, or a combination thereof.

14. The aqueous colloidal suspension of claim 11, wherein the base comprises at least one of sodium hydroxide, potassium hydroxide, strontium hydroxide, barium hydroxide, rubidium hydroxide, calcium hydroxide, ammonium hydroxide, magnesium hydroxide, lithium hydroxide, cesium hydroxide, DMSO, DMA, Trimethylphosphine, EtOAC, arginine, ammonia, trimethyl ammonia, pyridine, methylamine, alanine, or a combination thereof.

15. The aqueous colloidal suspension of claim 11, further comprising a pH buffer.

16. The aqueous colloidal suspension of claim 15, wherein the pH buffer comprises at least one of glycine, arginine, or a combination thereof.

17. The aqueous colloidal suspension of claim 15, wherein the pH buffer comprises at least one of ACES, ADA, ammonium hydroxide, 2-amino-2-methyl-1-propanol, AMPD (2 amino-2-methyl-1,3-propanediol), AMPSO, BES, BICINE, bis-tris, bis-tris propane, borate, boric acid, CABs, Cacodylate, CAPS, CAPSO, Carbonate (Salts included), CHES, Citrate (Salts included), DIPS, EPPS, HEPPS, Ethanolamine, Glycine, glycylglycine, HEPBS, HEPES, HEPPSO, histidine, hydrazine, imidazole, maleate, MES, methylamine, MOBS, MOPS, MOPSO, phosphate (Salts included), piperazine, piperidine, PIPES, POPSO, pyrophosphate, TABS, TAPS, TAPSO, taurine, TES, tricine, triethanolamine, trizma, or a combination thereof.
